(19) United States
(12) Patent Application Publication
(10) Pub. No.: US 2016/0317004 A1
(43) Pub. Date: Nov. 3, 2016

(54) IMAGING APPARATUS

(71) Applicant: OLYMPUS CORPORATION, Tokyo (JP)

(72) Inventor: Kazunori YOSHIZAKI, Tokyo (JP)

(73) Assignees: OLYMPUS CORPORATION, Tokyo (JP); EPAS

(21) Appl. No.: 14/977,319

(22) Filed: Dec. 21, 2015

Related U.S. Application Data
(63) Continuation of application No. PCT/JP2015/063016, filed on Apr. 30, 2015.

(51) Int. Cl. A61B 1/06; A61B 1/04; A61B 1/00; H04N 5/33; H04N 5/225

(52) U.S. Cl. CPC A61B 1/0646; H04N 5/332; H04N 5/2256; A61B 1/0661; A61B 1/0638; A61B 1/00006; A61B 1/00009; A61B 1/04

(57) ABSTRACT

An imaging apparatus includes: an imaging element; a filter array including a unit including visible light filters with different transmission spectrum maximum values, and invisible light filters having a transmission spectrum maximum value in an invisible light range of wavelengths longer than those of the visible light band; an optical filter disposed on a light-receiving surface of the filter array, the optical filter transmitting light included in either a first wavelength band that includes the respective transmission spectrum maximum values of the visible light filters or a second wavelength band that includes the transmission spectrum maximum value of the invisible light filters; and a first light source that emits, toward the subject, light having a wavelength within the second wavelength band, light of a first wavelength having a half-value width less than or equal to half of the second wavelength band.

### CROSS REFERENCES TO RELATED APPLICATIONS

This application is a continuation of International Application No. PCT/JP2015/063016, filed on Apr. 30, 2015, the entire contents of which are incorporated herein by reference.

### BACKGROUND OF THE INVENTION

1. Field of the Invention

The present invention relates to imaging apparatuses for imaging subjects and generating image data used for detecting vital information on the subjects.

2. Description of the Related Art

In the medical field, as information to determine the state of human health, vital information such as a heart rate, oxygen saturation, and blood pressure has been used to determine the state of a subject's health. For example, there is a known technology that images, by an image sensor, a living body such as a finger brought into contact with the inside of a measurement probe that emits red light and near-infrared light, separately, and calculates the oxygen saturation of the living body, based on image data generated by the image sensor (see Japanese Laid-open Patent Publication No. 2013-118978). According to this technology, the oxygen saturation of a living body is calculated, based on the degree of light absorption by the living body calculated according to image data generated by the image sensor, and changes in the degree of light absorption over time.

### SUMMARY OF THE INVENTION

An imaging apparatus according to one aspect of the present invention generates image data for detecting vital information on a subject and includes: an imaging element that generates the image data by photoelectrically converting light received by each of a plurality of pixels arranged two-dimensionally; a filter array including a unit including a plurality of visible light filters with different transmission spectrum maximum values within a visible light band, and invisible light filters having a transmission spectrum maximum value in an invisible light range of wavelengths longer than those of the visible light band, the visible light filters and the invisible light filters being disposed in correspondence with the plurality of pixels; an optical filter disposed on a light-receiving surface of the filter array, the optical filter transmitting light included in either a first wavelength band that includes the respective transmission spectrum maximum values of the plurality of visible light filters or a second wavelength band that includes the transmission spectrum maximum value of the invisible light filters; and a first light source that emits, toward the subject, light having a wavelength within the second wavelength band, light of a first wavelength having a half-value width less than or equal to half of the second wavelength band.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram illustrating a functional configuration of an imaging apparatus according to a first embodiment of the present invention;

FIG. 2 is a diagram schematically illustrating a configuration of a filter array according to the first embodiment of the present invention;

FIG. 3 is a graph illustrating an example of the transmittance characteristics of each filter according to the first embodiment of the present invention;

FIG. 4 is a graph illustrating the relationship between the transmittance characteristics of an optical filter and light of a first wavelength emitted by a first light source according to the first embodiment of the present invention;

FIG. 5 is a block diagram illustrating a functional configuration of an imaging apparatus according to a second embodiment of the present invention;

FIG. 6 is a graph illustrating the relationship between the transmittance characteristics of an optical filter of the imaging apparatus and light of a first wavelength emitted by a first light source and light of a second wavelength emitted by a second light source according to the second embodiment of the present invention;

FIGS. 7A and 7B are diagrams illustrating timing charts of light-emission timings for the first light source and the second light source controlled by an illumination control unit of the imaging apparatus according to the second embodiment of the present invention;

FIGS. 8A and 8B are diagrams illustrating timing charts of light-emission timings for the first light source and the second light source controlled by the illumination control unit of the imaging apparatus according to a modification of the second embodiment of the present invention;

FIG. 9 is a block diagram illustrating a functional configuration of an imaging apparatus according to a third embodiment of the present invention;

FIG. 10 is a diagram schematically illustrating a configuration of a filter array of the imaging apparatus according to the third embodiment of the present invention;

FIG. 11 is a graph illustrating an example of the transmittance characteristics of each filter of the imaging apparatus according to the third embodiment of the present invention;

FIG. 12 is a graph illustrating an example of the transmittance characteristics of an optical filter of the imaging apparatus according to the third embodiment of the present invention;

FIGS. 13A and 13B are diagrams illustrating timing charts of light-emission timings for a first light source and a second light source controlled by an illumination control unit of the imaging apparatus according to the third embodiment of the present invention; and

FIG. 14 is a graph illustrating hemoglobin absorption characteristics in the blood.

### DETAILED DESCRIPTION OF THE PREFERRED EMBODIMENTS

Hereinafter, embodiments to implement the present invention will be described in detail with the drawings. The embodiments below are not intended to limit the present invention. The drawings referred to in the description below only approximately illustrate shapes, sizes, and positional relationships to the extent that details of the present invention can be understood. That is, the present invention is not limited only to the shapes, sizes, and positional relationships illustrated in the drawings. The same components are denoted by the same reference numerals in the description.

### First Embodiment

Configuration of Imaging Apparatus

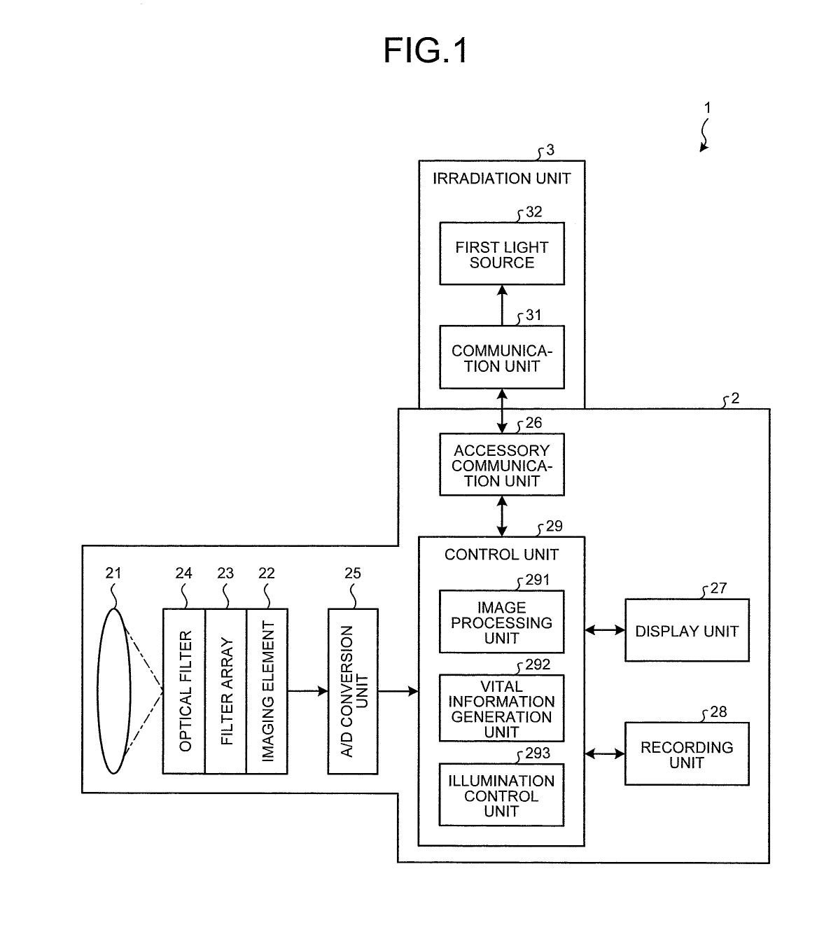

FIG. 1 is a block diagram illustrating a functional configuration of an imaging apparatus according to a first embodiment of the present invention. An imaging apparatus 1 illustrated in FIG. 1 includes a main body 2 that images a subject and generates image data on the subject, and an irradiation unit 3 that is detachably attached to the main body 2 and emits light having a predetermined wavelength band toward an imaging area of the imaging apparatus 1.

Configuration of Main Body

First, a configuration of the main body 2 will be described.

The main body 2 includes an optical system 21, an imaging element 22, a filter array 23, an optical filter 24, an A/D conversion unit 25, an accessory communication unit 26, a display unit 27, a recording unit 28, and a control unit (a controller or a processor) 29.

The optical system 21 is configured using one or a plurality of lenses such as a focus lens and a zoom lens, a diaphragm, and a shutter, or the like, to form a subject image on a light-receiving surface of the imaging element 22.

The imaging element 22 receives light of a subject image that has passed through the optical filter 24 and the filter array 23, and performs photoelectric conversion, thereby generating image data continuously according to a predetermined frame (e.g. 60 fps). The imaging element 22 is configured using a complementary metal oxide semiconductor (CMOS), a charge coupled device (CCD), or the like, which photoelectrically converts light that has passed through the optical filter 24 and the filter array 23 and received by each of a plurality of pixels arranged two-dimensionally, and generates electrical signals.

The filter array 23 is disposed on the light-receiving surface of the imaging element 22. The filter array 23 has a unit including a plurality of visible light filters with different transmission spectrum maximum values within a visible light band, and invisible light filters having a transmission spectrum maximum value in an invisible light range of wavelengths longer than those of a visible light range, disposed in correspondence with the plurality of pixels in the imaging element 22.

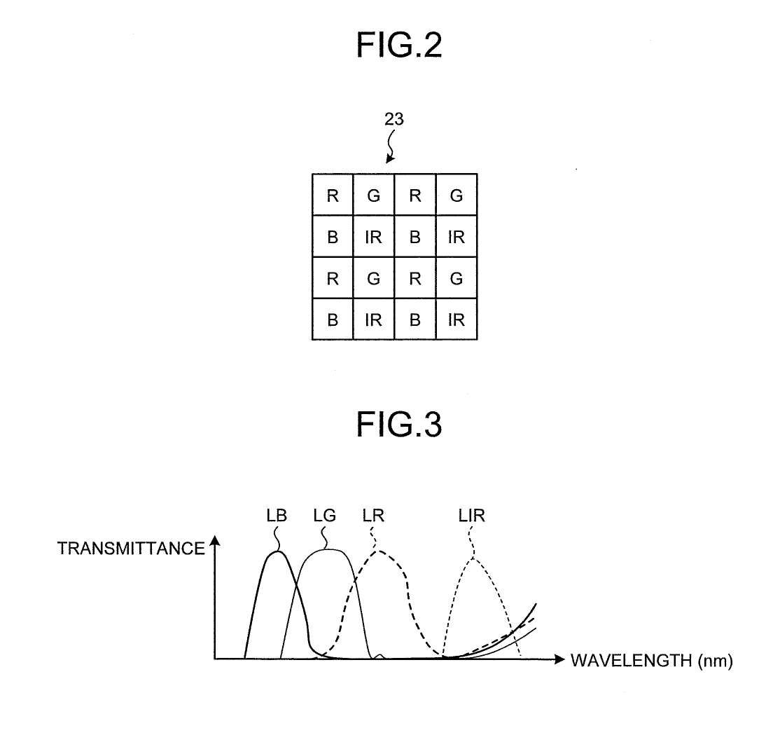

FIG. 2 is a diagram schematically illustrating a configuration of the filter array 23. As illustrated in FIG. 2, the filter array 23 is disposed on respective light-receiving surfaces of the pixels constituting the imaging element 22, and has a unit including visible light filters R that transmit red light, visible light filters G that transit green light, visible light filters B that transmit blue light, and invisible light filters IR that transmit invisible light, disposed in correspondence with the plurality of pixels. Hereinafter, a pixel on which a visible light filter R is disposed is described as an R pixel, a pixel on which a visible light filter G is disposed as a G pixel, a pixel on which a visible light filter B is disposed as a B pixel, and a pixel on which an invisible light filter IR is disposed as an IR pixel.

FIG. 3 is a graph illustrating an example of the transmittance characteristics of each filter. In FIG. 3, the horizontal axis represents wavelength (nm), and the vertical axis represents transmittance. In FIG. 3, a curved line LR represents the transmittance of the visible light filters R, a curved line LG represents the transmittance of the visible light filters G, a curved line LB represents the transmittance of the visible light filters B, and a curved line LIR represents the transmittance of the invisible light filters IR. In FIG. 3, although the transmittance characteristics of each filter are illustrated to simplify the description, they are equal to the spectral sensitivity characteristics of each pixel (R pixels, G pixels, B pixels, and IR pixels) when each pixel is provided with a respective filter.

As illustrated in FIG. 3, the visible light filters R have a transmission spectrum maximum value in a visible light band. Specifically, the visible light filters R have the transmission spectrum maximum value in a wavelength band of 620 to 750 nm, and transmit light of the wavelength band of 620 to 750 nm, and also transmit part of light of a wavelength band of 850 to 950 nm in an invisible light range. The visible light filters G have a transmission spectrum maximum value in the visible light band. Specifically, the visible light filters G have the transmission spectrum maximum value in a wavelength band of 495 to 570 nm, and transmit light of the wavelength band of 495 to 570 nm, and also transmit part of light of the wavelength band of 850 to 950 nm in the invisible light range. The visible light filters B have a transmission spectrum maximum value in the visible light band. Specifically, the visible light filters B have the transmission spectrum maximum value in a wavelength band of 450 to 495 nm, and transmit light of the wavelength band of 450 to 495 nm, and also transmit part of light of the wavelength band of 850 to 950 nm in the invisible light range. The invisible light filters IR have a transmission spectrum maximum value in an invisible light band, and transmit light of the wavelength band of 850 to 950 nm.

Returning to FIG. 1, description of the configuration of the main body 2 will be continued.

The optical filter 24 is disposed at the front of the filter array 23, and transmits light having a wavelength included in either a first wavelength band including the respective transmission spectrum maximum values of the visible light filters R, the visible light filters G, and the visible light filters B, or a second wavelength band including the transmission spectrum maximum value of the invisible light filters IR.

The A/D conversion unit 25 converts analog image data input from the imaging element 22 to digital image data, and outputs it to the control unit 29.

The accessory communication unit 26 transmits a drive signal to an accessory connected to the main body 2, under the control of the control unit 29, in compliance with a predetermined communication standard.

The display unit 27 displays images corresponding to image data input from the control unit 29. The display unit 27 is configured using a liquid crystal or organic electro luminescence (EL) display panel, or the like.

The recording unit 28 records various kinds of information on the imaging apparatus 1. The recording unit 28 records image data generated by the imaging element 22, various programs for the imaging apparatus 1, parameters for processing being executed, and the like. The recording unit 28 is configured using synchronous dynamic random access memory (SDRAM), flash memory, a recording medium, or the like.

The control unit 29 performs instructions, data transfer, and so on to units constituting the imaging apparatus 1, thereby centrally controlling the operation of the imaging apparatus 1. The control unit 29 is configured using a central processing unit (CPU), a processor or the like.

Here, a detailed configuration of the control unit 29 will be described. The control unit 29 includes at least an image processing unit (an image processor) 291, a vital information generation unit 292, and an illumination control unit 293.

The image processing unit 291 performs predetermined image processing on image data input from the A/D conversion unit 25. Here, the predetermined image processing includes optical black subtraction processing, white balance adjustment processing, image data synchronization processing, color matrix arithmetic processing, γ correction processing, color reproduction processing, and edge enhancement processing.

The vital information generation unit 292 generates vital information on a subject, based on image signals corresponding to IR pixels included in image data that is input continuously from the A/D conversion unit 25. Here, the vital information is at least one of oxygen saturation, a heart rate, heart rate variability, stress, skin moisture, and a blood pressure.

The illumination control unit 293 controls light emission of the irradiation unit 3 connected to the main body 2 via the accessory communication unit 26. For example, in a case where a vital information generation mode to generate vital information on a subject is set in the imaging apparatus 1, when the irradiation unit 3 is connected to the main body 2, the illumination control unit 293 causes the irradiation unit 3 to emit light in synchronization with imaging timing of the imaging element 22.

Configuration of Irradiation Unit

Next, a configuration of the irradiation unit 3 will be described. The irradiation unit 3 includes a communication unit 31 and a first light source 32.

The communication unit 31 outputs a drive signal input from the accessory communication unit 26 of the main body 2 to the first light source 32.

According to a drive signal input from the main body 2 via the communication unit 31, the first light source 32 emits, toward a subject, light having a wavelength within the second wavelength band that is transmitted by the optical filter 24, light of a first wavelength having a half-value width less than or equal to half of the second wavelength band (hereinafter, referred to as “first wavelength light”). The first light source 32 is configured using a light emitting diode (LED).

The imaging apparatus 1 configured like this images a subject, irradiating it with the first wavelength light, thereby generating color image data (respective image signals of the R pixels, G pixels, and B pixels) and image data to obtain vital information (image signals of the IR pixels (near-infrared image data)) on the subject.

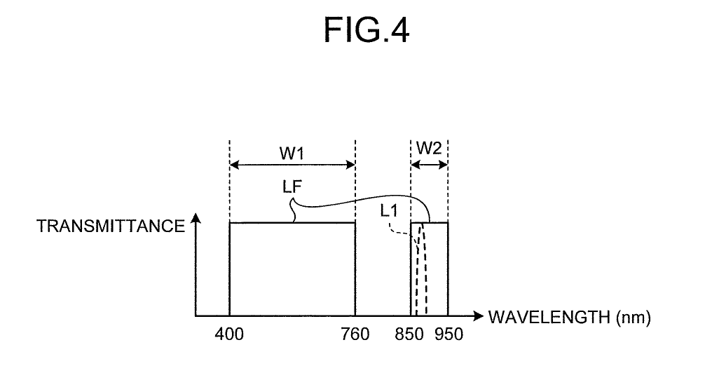

Next, the relationship between the above-described optical filter 24 and the first wavelength light emitted by the first light source 32 will be described. FIG. 4 is a graph illustrating the relationship between the transmittance characteristics of the optical filter 24 and the first wavelength light emitted by the first light source 32. In FIG. 4, the horizontal axis represents wavelength (nm), and the vertical axis represents transmittance. In FIG. 4, a broken line LF represents the transmittance characteristics of the optical filter 24, and a curved line L1 represents the wavelength band of the first wavelength light emitted by the first light source 32.

As illustrated in FIG. 4, the optical filter 24 only transmits light having a wavelength included in either a first wavelength band W1 including the respective transmission spectra of the visible light filters R, the visible light filters G, and the visible light filters B, or a second wavelength band W2 of the transmission spectrum of the invisible light filters IR. Specifically, the optical filter 24 transmits light of 400 to 760 nm in the visible light range, and transmits light of 850 to 950 nm in the invisible light range. As shown by the curved line L1, the first light source 32 emits the first wavelength light that is within the second wavelength band W2 in the optical filter 24 and has a half-value width less than or equal to half of the second wavelength band W2. Specifically, the first light source 32 emits light of 860 to 900 nm. Thus, color image data on visible light and image data on invisible light to obtain vital information can each be obtained. In FIG. 4, in order to simplify the description, the optical filter 24 transmits light of 400 to 760 nm in the visible light range, and transmits light of 850 to 950 nm in the invisible light range. As a matter of course, it may alternatively allow at least part of light having a wavelength band of 760 to 850 nm to pass through (not allow at least part of that to pass through). For example, the optical filter 24 may allow light having at least part of a wavelength band of 770 to 800 nm to pass through.

According to the above-described first embodiment of the present invention, the first light source 32 emits the first wavelength light that is within the second wavelength band W2 in the optical filter 24 and has a half-value width less than or equal to half of the second wavelength band W2, so that image data to generate vital information on a subject can be obtained in a non-contact state.

Further, according to the first embodiment of the present invention, the optical filter 24 transmits light having a wavelength including either the first wavelength band including the respective transmission spectra of the visible light filters R, the visible light filters G, and the visible light filters B, or the second wavelength band including the transmission spectrum of the invisible light filters IR, thereby removing unnecessary information (wavelength components), so that an improvement in the accuracy of the visible light range can be realized (higher resolution), and the degree of freedom in an optical source used for the invisible light range can be improved.

Although the first light source 32 emits light of 860 to 900 nm as the first wavelength light in the first embodiment of the present invention, it may be configured using an LED capable of emitting light of 970 nm when skin moisture is detected as vital information on a living body, for example. At this time, the optical filter 24 capable of transmitting light of a visible light band of 900 to 1000 nm as the second wavelength band may be used.

In the first embodiment of the present invention, the vital information generation unit 292 may detect skin color variability of an subject, based on image signals from the IR pixels in image data of the imaging element 22 input continuously from the A/D conversion unit 25 (hereinafter, referred to as “moving image data”), detect a heart rate/heart rate variability of the subject, based on respective image signals of the R pixels, the G pixels, and the B pixels in the moving image data, and detect an accurate heart rate of the subject, based on the detected heart rate/heart rate variability and the above-described skin color variability of the subject. Further, the vital information generation unit 292 may detect the degree of stress of the subject from a waveform of the above-described heart rate variability, as vital information.

Although the irradiation unit 3 is detachably attached to the main body 2 in the first embodiment of the present invention, the irradiation unit 3 and the main body 2 may be formed integrally.

### Second Embodiment

Next, a second embodiment of the present invention will be described. An imaging apparatus according to the second embodiment is different in configuration from the imaging apparatus 1 according to the above-described first embodiment. Specifically, the imaging apparatus according to the second embodiment is different in the configuration of the irradiation unit 3 of the imaging apparatus 1 according to the above-described first embodiment. Thus, hereinafter, after the configuration of an irradiation unit of the imaging apparatus according to the second embodiment is described, processing executed by the imaging apparatus according to the second embodiment will be described. The same components as those of the imaging apparatus 1 according to the above-described first embodiment are denoted by the same reference numerals and will not be described.

Configuration of Imaging Apparatus

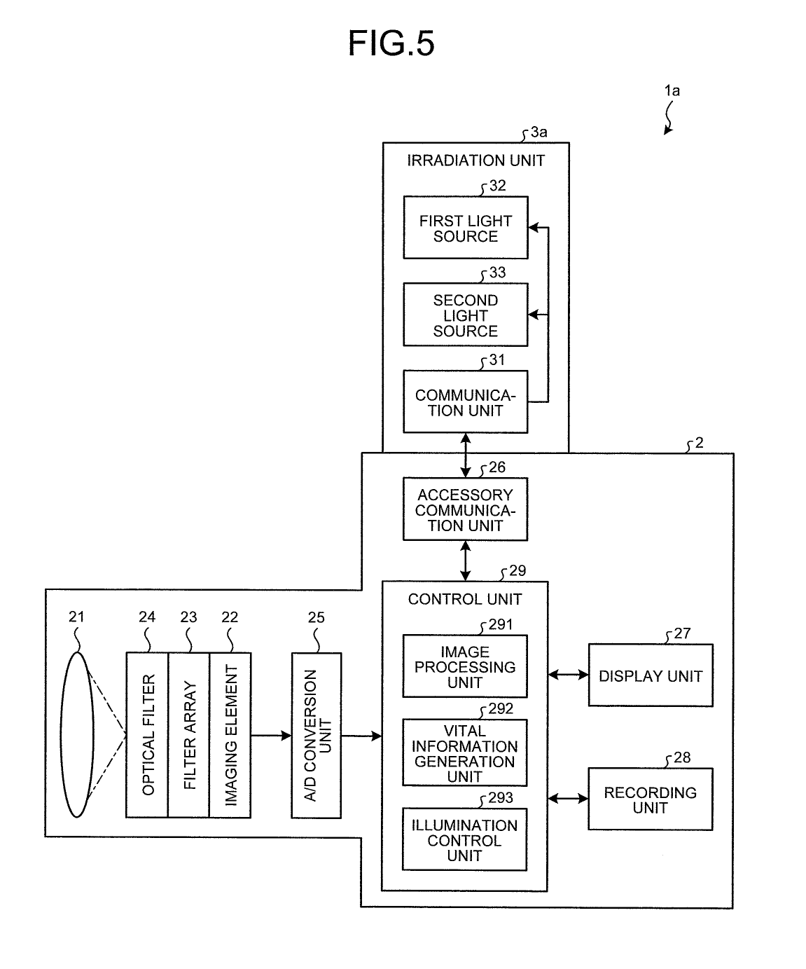

FIG. 5 is a block diagram illustrating a functional configuration of an imaging apparatus according to the second embodiment of the present invention. An imaging apparatus la illustrated in FIG. 5 includes a main body 2 and an irradiation unit 3a in place of the irradiation unit 3 of the imaging apparatus 1 according to the above-described first embodiment.

Configuration of Irradiation Unit

The irradiation unit 3a emits light having a predetermined wavelength band toward an imaging area of the imaging apparatus 1a. The irradiation unit 3a further includes a second light source 33 in addition to the configuration of the irradiation unit 3 according to the above-described first embodiment.

The second light source 33 emits, toward a subject, light having a wavelength within a second wavelength band in an optical filter 24, light of a second wavelength having a half-value width less than or equal to half of the second wavelength band, which is different from light of a first wavelength (hereinafter, referred to as “second wavelength light”). The second light source 33 is configured using an LED.

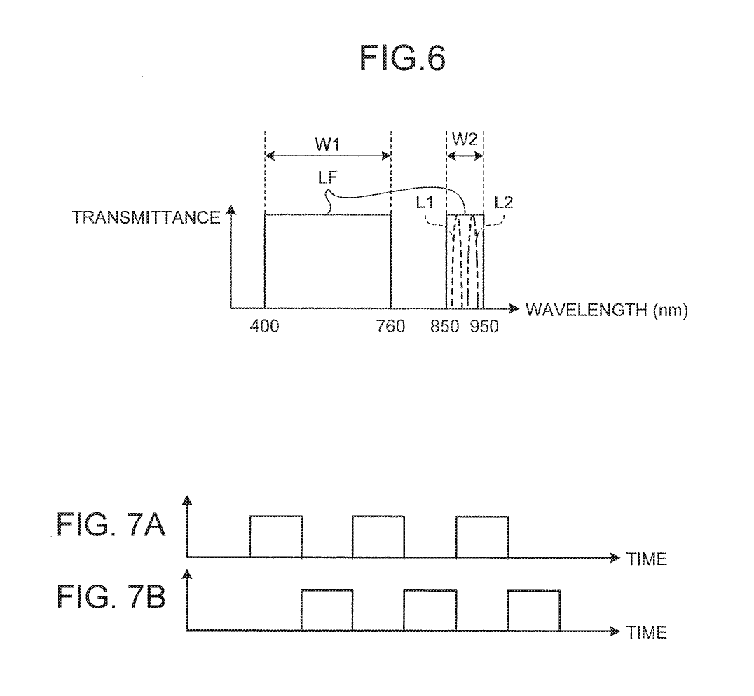

Next, the relationship between the above-described optical filter 24 and light of the first wavelength band emitted by a first light source 32 and light of the second wavelength band emitted by the second light source 33 will be described. FIG. 6 is a graph illustrating the relationship between the transmittance characteristics of the optical filter 24 and light of the first wavelength band emitted by the first light source 32 and light of the second wavelength band emitted by the second light source 33. In FIG. 6, the horizontal axis represents wavelength (nm), and the vertical axis represents transmittance. In FIG. 6, a broken line LF represents the transmittance characteristics of the optical filter 24, a curved line L1 represents the wavelength band of the first wavelength light emitted by the first light source 32, and a curved line L2 represents the wavelength band of the second wavelength light emitted by the second light source 33.

As illustrated in FIG. 6, the optical filter 24 transmits light having a wavelength including either respective light of a first wavelength band W1 of visible light filters R, visible light filters G, and visible light filters B, or a second wavelength band W2 of invisible light filters IR. As shown by the curved line L1, the first light source 32 emits the first wavelength light that is within the second wavelength band transmitted by the optical filter 24 and has a half-value width less than or equal to half of the second wavelength band. Further, as shown by the curved line L2, the second light source 33 emits the second wavelength light that is within the second wavelength band transmitted by the optical filter 24 and has a half-value width less than or equal to half of the second wavelength band. Further, the second light source 33 emits the second wavelength light having a wavelength band different from a first wavelength band of light emitted by the first light source 32. Specifically, the second light source 33 emits light of 900 to 950 nm.

Processing by Illumination Control Unit

Next, light emission timings for the first light source 32 and the second light source 33 controlled by the illumination control unit 293 will be described. FIGS. 7A and 7B are diagrams illustrating timing charts of light emission timings for the first light source 32 and the second light source 33 controlled by the illumination control unit 293. In FIGS. 7A and 7B, the horizontal axis represents time. FIG. 7A illustrates light emission timings for the first light source 32, and FIG. 7B illustrates light emission timings for the second light source 33.

As illustrated in FIGS. 7A and 7B, the illumination control unit 293 causes the first light source 32 and the second light source 33 to emit light alternately via an accessory communication unit 26 and a communication unit 31, thereby irradiating a subject with the first wavelength light and the second wavelength light in a time-division manner. This allows the obtainment of information on the second wavelength light in addition to that on the first wavelength light.

According to the second embodiment of the present invention described above, the second light source 33 to emit, toward a subject, light within the second wavelength band in the optical filter 24, the second wavelength light having a half-value width less than or equal to half of the second wavelength band, which is different from the first wavelength light, is further provided, and the illumination control unit 293 causes the first light source 32 and the second light source 33 to emit light alternately, so that vital information can be obtained, and also space information and distance information on a three-dimensional map produced by 3D pattern projection can be obtained.

### Modification of Second Embodiment

Although the illumination control unit 293 causes the first light source 32 and the second light source 33 to emit light alternately in the second embodiment of the present invention, light emission timings may be changed at intervals of a predetermined number of frames of image data generated by the imaging element 22, for example.

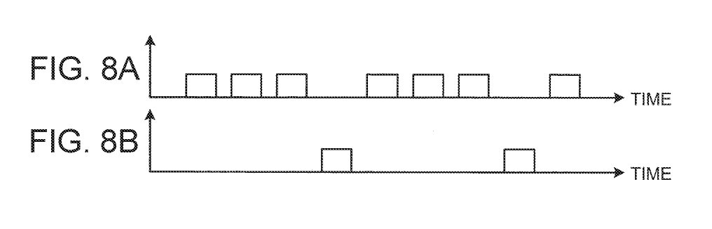

FIGS. 8A and 8B are diagrams illustrating timing charts of light emission timings for the first light source 32 and the second light source 33 controlled by the illumination control unit 293 according to a modification of the second embodiment of the present invention. In FIGS. 8A and 8B, the horizontal axis represents time. FIG. 8A illustrates light emission timings for the first light source 32, and FIG. 8B illustrates light emission timings for the second light source 33.

As illustrated in FIGS. 8A and 8B, the illumination control unit 293 causes the first light source 32 and the second light source 33 to emit light in a predetermined pattern with the first light source 32 synchronized with a frame rate of the imaging element 22 via the accessory communication unit 26 and the communication unit 31. Specifically, the illumination control unit 293 causes the first light source 32 to emit light a predetermined number of times, e.g. three times, and thereafter causes the second light source 33 to emit light once. This allows the obtainment of information on the second wavelength light in addition to that on the first wavelength light.

According to the modification of the second embodiment of the present invention described above, vital information can be obtained, and also space information and distance information on a three-dimensional map produced by 3D pattern projection can be obtained.

Although the illumination control unit 293 changes light emission timings at intervals of the number of frames of the imaging element 22 in the modification of the second embodiment of the present invention, light emission time of the first light source 32 and the second light source 33 may be changed, for example. Specifically, the illumination control unit 293 may be caused to repeatedly execute an operation of causing the first light source 32 to emit light for a first predetermined period of time, e.g. thirty seconds, and thereafter causing the second light source 33 to emit light for a second predetermined period of time shorter than the first predetermined period of time, e.g. five seconds.

### Third Embodiment

Next, a third embodiment of the present invention will be described. An imaging apparatus according to the third embodiment is different in configuration from the imaging apparatus la according to the above-described second embodiment. Specifically, the imaging apparatus according to the third embodiment is different in the configuration of a color filter. Thus, hereinafter, after the imaging apparatus according to the third embodiment is described, processing executed by the third embodiment will be described. The same components as those of the imaging apparatus la according to the above-described second embodiment are denoted by the same reference numerals and will not be described.

Configuration of Imaging Apparatus

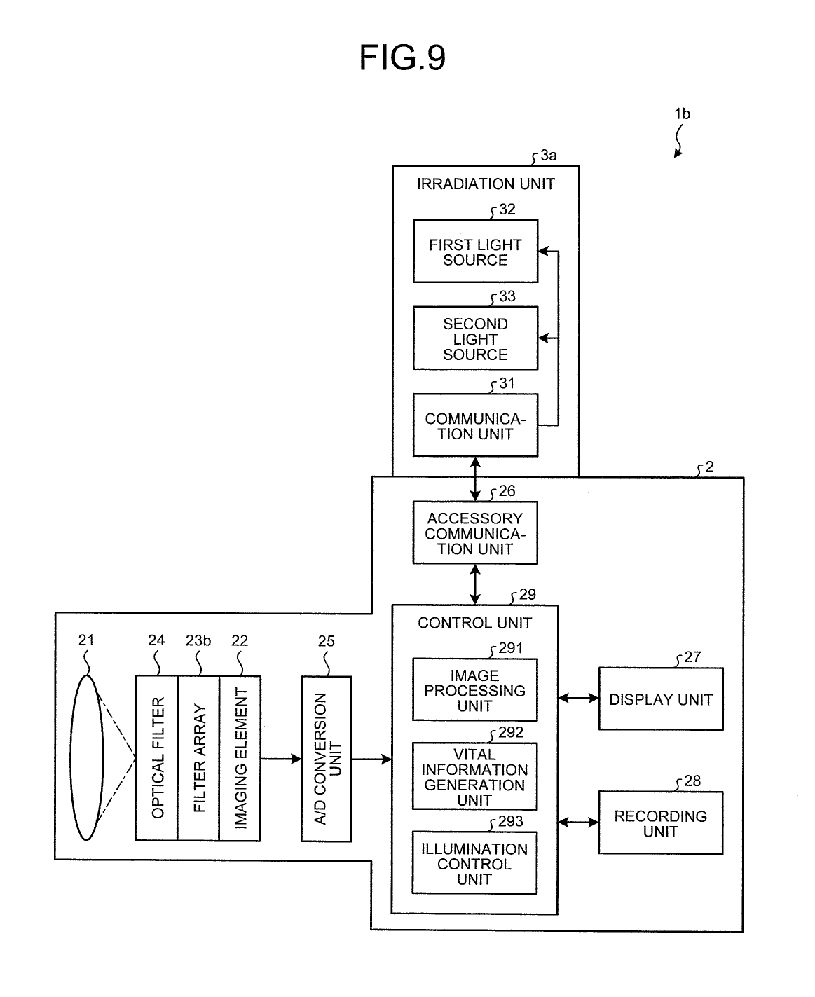

FIG. 9 is a block diagram illustrating a functional configuration of an imaging apparatus according to the third embodiment of the present invention. An imaging apparatus 1b illustrated in FIG. 9 includes a filter array 23b in place of the filter array 23 of the imaging apparatus 1a according to the above-described second embodiment.

The filter array 23b includes a plurality of visible light filters with different transmission spectrum maximum values within a visible light band, and a plurality of invisible light filters with different transmission spectrum maximum values within an invisible light range, an invisible light range of wavelengths longer than those of a visible light range.

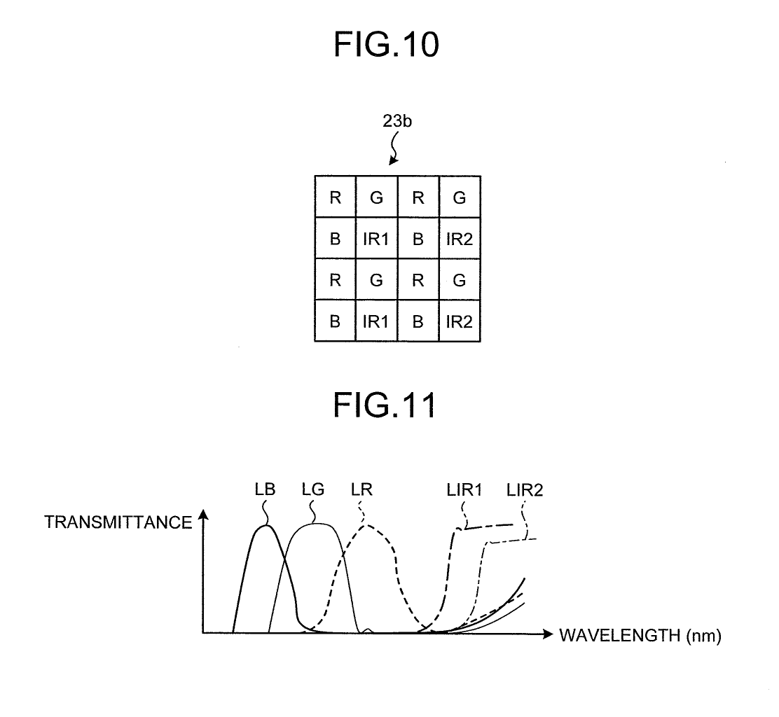

FIG. 10 is a diagram schematically illustrating a configuration of the filter array 23b. As illustrated in FIG. 10, the filter array 23b has a unit including visible light filters R, visible light filters G, visible light filters B, first invisible light filters IR1 that transmit light of invisible light, and second invisible light filters IR2 that transmit light of invisible light different from that of the first invisible light filters IR1, disposed in correspondence with a plurality of pixels. Hereinafter, a pixel on which a first invisible light filter IR1 is disposed is described as a first IR pixel, and a pixel on which a second invisible light filter IR2 is disposed as a second IR pixel.

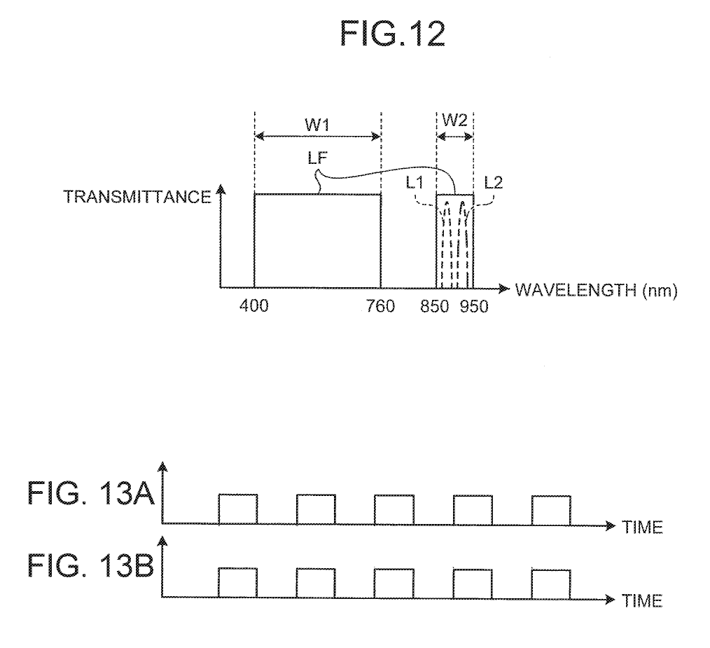

FIG. 11 is a graph illustrating an example of the transmittance characteristics of each filter. FIG. 12 is a graph illustrating an example of the transmittance characteristics of the optical filter 24. In FIGS. 11 and 12, the horizontal axis represents wavelength (nm), and the vertical axis represents transmittance. In FIG. 11, a curved line LR represents the transmittance of the visible light filters R, a curved line LG represents the transmittance of the visible light filters G, a curved line LB represents the transmittance of the visible light filters B, a curved line LIR1 represents the transmittance of the first invisible light filters IR1, and a curved line LIR2 represents the transmittance of the second invisible light filters IR2.

As illustrated in FIGS. 11 and 12, the first invisible light filters IR1 have a transmission spectrum maximum value in an invisible light band, and transmit light of a wavelength band of 850 to 950 nm. The second invisible light filters IR2 have a transmission spectrum maximum value in the invisible light band, and transmits light of a wavelength band of 850 to 950 nm.

Processing by Illumination Control Unit

Next, light emission timings for the first light source 32 and the second light source 33 controlled by the illumination control unit 293 will be described. FIGS. 13A and 13B are diagrams illustrating timing charts of light emission timings for the first light source 32 and the second light source 33 controlled by the illumination control unit 293. In FIGS. 13A and 13B, the horizontal axis represents time. FIG. 13A illustrates light emission timings for the first light source 32, and FIG. 13B illustrates light emission timings for the second light source.

As illustrated in FIGS. 13A and 13B, the illumination control unit 293 causes the first light source 32 and the second light source 33 to emit light simultaneously via an accessory communication unit 26 and a communication unit 31, thereby irradiating a subject with first wavelength light and second wavelength light simultaneously. This allows the simultaneous obtainment of information on the first wavelength light and that on the second wavelength light.

According to the third embodiment of the present invention described above, since the illumination control unit 293 causes the first light source 32 and the second light source 33 to emit light simultaneously, vital information and space information and distance information on a three-dimensional map produced by 3D pattern projection can be obtained simultaneously.

### Modification of the Third Embodiment

In the third embodiment of the present invention, vital information and space information and distance information on a three-dimensional map produced by 3D pattern projection are obtained simultaneously. As the vital information, oxygen saturation in the blood may be obtained.

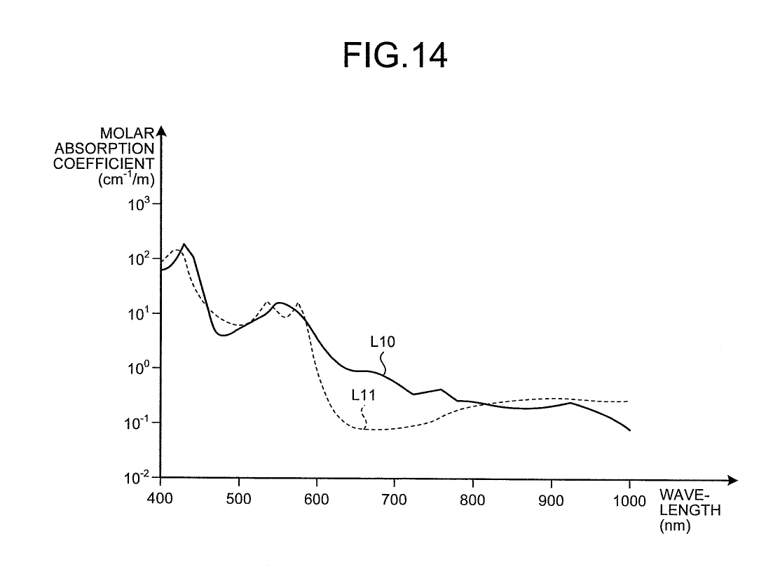

FIG. 14 is a graph illustrating hemoglobin absorption characteristics in the blood. In FIG. 14, the horizontal axis represents wavelength (nm), and the vertical axis represents molar absorption coefficient (cm⁻¹/m). In FIG. 14, a curved line L10 represents the molar absorption coefficient of oxygenated hemoglobin, and a curved line L11 represents the molar absorption coefficient of deoxygenated hemoglobin.

There are two types of blood hemoglobin, deoxygenated hemoglobin (Hb), which is not combined with oxygen, and oxygenated hemoglobin (HbO₂), which is combined with oxygen. Oxygen saturation (SPO₂) used in the modification of the third embodiment represents the proportion of oxygenated hemoglobin in all hemoglobin in the blood. The oxygen saturation is calculated by the following expression (1):

SPO₂=(C((HbO₂)/(C(HbO₂)+(C(Hb)))×100 (1)

wherein C ((HbO₂) represents the concentration of oxygenated hemoglobin, and (C(Hb)) represents the concentration of deoxygenated hemoglobin.

In the modification of the third embodiment, differences in the respective absorption characteristics at each wavelength between oxygenated hemoglobin and deoxygenated hemoglobin are used. Specifically, as illustrated in FIG. 14, in the modification of the third embodiment, the first light source 32 emits light of 940 nm in a near-infrared range, and the second light source 33 emits light of 1000 nm in an infrared range, and a vital information generation unit 292 calculates oxygen saturation, based on respective image signals of the first IR pixels and the second IR pixels included in image data (see Japanese Laid-open Patent Publication No. 2013-118978 for a theoretical method for oxygen saturation. Or see Lingqin Kong et al., “Non-contact detection of oxygen saturation based on visible light imaging device using ambient light,” Optics Express, Vol. 21, Issue 15, pp. 17464-17471 (2013) for a method for oxygen saturation by non-contact (a method for indirect estimation using image data)).

According to the modification of the third embodiment of the present invention described above, as vital information, oxygen saturation in the blood can be detected in a non-contact manner.

### Other Embodiments

Although in the above-described first to third embodiments, the first light source or the second light source is configured using an LED, it may alternatively be configured using a light source that emits light of a visible light wavelength band and a near-infrared wavelength band like a halogen light source, for example.

Although in the above-described first to third embodiments, as visible light filters, primary color filters, such as the visible light filters R, the visible light filters G, and the visible light filters B, are used, complementary color filters such as magenta, cyan, and yellow, for example, may alternatively be used.

Although in the above-described first to third embodiments, the optical system, the optical filter, the filter array, and the imaging element are built into the main body, the optical system, the optical filter, the filter array, and the imaging element may alternatively be housed in a unit, and the unit may be detachably attached to the main body. As a matter of course, the optical system may be housed in a lens barrel, and the lens barrel may be configured to be detachably attached to a unit housing the optical filter, the filter array, and the imaging element.

In the above-described first to third embodiments, the vital information generation unit is provided in the main body. Alternatively, for example, a function capable of generating vital information may be actualized by a program or application software in a mobile device or a wearable device such as a watch or glasses capable of bidirectional communication, and by transmitting image data generated by an imaging apparatus, the mobile device or the wearable device may generate vital information on a subject.

The present invention is not limited to the above-described embodiments, and various modifications and applications may be made within the gist of the present invention, as a matter of course. For example, other than the imaging apparatus used to describe the present invention, the present invention can be applied to any apparatus capable of imaging a subject, such as a mobile device or a wearable device equipped with an imaging element in a mobile phone or a smartphone, or an imaging apparatus for imaging a subject through an optical device, such as a video camera, an endoscope, a surveillance camera, or a microscope.

A method of each processing by the imaging apparatus in the above-described embodiments, that is, processing illustrated in each timing chart may each be stored as a program that a control unit such as a CPU can be caused to execute. Besides, it can be stored in a storage medium of an external storage device such as a memory card (such as a ROM card or a RAM card), a magnetic disk, an optical disk (such as a CD-ROM or a DVD), or semiconductor memory for distribution. The control unit such as a CPU reads the program stored in the storage medium of the external storage device, and by the operation being controlled by the read program, the above-described processing can be executed.

According to the above-described Embodiments, it is possible to obtain vital information on a living body in a non-contact state.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An imaging apparatus that generates image data for detecting vital information on a subject, the apparatus comprising:
an imaging element that generates the image data by photoelectrically converting light received by each of a plurality of pixels arranged two-dimensionally;
a filter array including a unit including a plurality of visible light filters with different transmission spectrum maximum values within a visible light band, and invisible light filters having a transmission spectrum maximum value in an invisible light range of wavelengths longer than those of the visible light band, the visible light filters and the invisible light filters being disposed in correspondence with the plurality of pixels;
an optical filter disposed on a light-receiving surface of the filter array, the optical filter transmitting light included in either a first wavelength band that includes the respective transmission spectrum maximum values of the plurality of visible light filters or a second wavelength band that includes the transmission spectrum maximum value of the invisible light filters; and
a first light source that emits, toward the subject, light having a wavelength within the second wavelength band, light of a first wavelength having a half-value width less than or equal to half of the second wavelength band.

2. The imaging apparatus according to claim 1, further comprising:
a second light source that emits, toward the subject, light having a wavelength within the second wavelength band, light of a second wavelength having a half-value width less than or equal to half of the second wavelength band, the light of the second wavelength being different from the light of the first wavelength; and
an illumination control unit that controls respective irradiation timings for the first light source and the second light source.

3. The imaging apparatus according to claim 2, wherein the illumination control unit causes the first light source and the second light source to emit light alternately in a predetermined pattern.

4. The imaging apparatus according to claim 2, wherein
the invisible light filters include:
a first invisible light filter that transmits light of the first wavelength; and
a second invisible light filter that transmits light of the second wavelength, and
the illumination control unit causes the first light source and the second light source to emit light simultaneously.

5. The imaging apparatus according to claim 3, wherein the first light source and the second light source are detachably attached to a main body of the imaging apparatus.

6. The imaging apparatus according to claim 1, further comprising a vital information generation unit that generates the vital information using the image data generated by the imaging element.
